(19) United States
(12) Patent Application Publication
(10) Pub. No.: US 2021/0127980 A1
(43) Pub. Date: May 6, 2021

(54) AN INTRAORAL MEDICAL DEVICE FOR PREDICTING ABNORMALITIES IN ORAL CANCER, MALIGNANT DISORDERS, INTERDENTAL CARIES, AND PERIDONTAL POCKETS

(71) Applicant: Rama Raju DEVARAJU, Hyderabad (IN)

(72) Inventors: Rama Raju DEVARAJU, Hyderabad (IN); Sree Surya YELURI, Houston, TX (US)

(21) Appl. No.: 16/639,731

(22) PCT Filed: Aug. 16, 2018
(86) PCT No.: PCT/IB2018/056160

§ 371 Date: Feb. 17, 2020

(30) Foreign Application Priority Data
Aug. 16, 2017 (IN) 201741028952

(51) Int. Cl. A61B 5/00

(52) U.S. Cl. CPC A61B 5/0088; A61B 5/0077; A61B 5/0004; A61B 2560/0431; A61B 5/0075; A61B 2560/0214; A61B 5/0022

(57) ABSTRACT

A hand held medical device with light emitting source and CCD camera for use to view upon oral cavity for intraoral screening of diseases such as oral cancer and potentially malignant disorders (PMD). The device has a supply of an illumination source of various wavelengths, a selector switch that enables the activation of a specific wavelength of light from the illumination source, a switch to adjust the intensity of various wavelength of the light emitting from the illumination source, an electronic system to control the selector switch and the illumination source, a camera to transmit and capture stored and live images from the oral cavity.

### CROSS REFERENCE TO RELATED APPLICATION AND CLAIM OF PRIORITY

The present application claims priority from Indian Patent Application No. 201741028952, filed on 16 Aug. 2017, the entire contents of which are hereby incorporated by reference.

### FIELD OF THE INVENTION

The present invention relates to an intraoral medical device (“DR Oroscope”) for diagnosing abnormalities in oral cavity. More particularly, the present invention relates to introduce an economic fluorescent visualization device using various wavelengths of light to detect abnormal oral tissues that cannot be seen by natural white light. The device will also assist in detection of tissue vasculature and infections in oral cavity.

### BACKGROUND OF THE INVENTION

Spectroscopic devices and endoscopes are being widely used as imaging devices based on tissue optics for screening and diagnostic purposes. Tissue optics refers to the effect of light on living tissue. In the identification of oral cancers blue light (wavelength 400-490 nm) has been used to identify abnormal tissues via fluorescence visualization loss (FVL). This technique is particularly useful as blue light penetrates about 1 mm deep, and over 95% of all oral cancers are squamous cell carcinomas. The thickness of the epithelium ranges from 99 um at the floor of the mouth to 294 um in the buccal mucosa, so blue light of 400-490 nm can easily reach this layer of the skin.

Tumors tend to exhibit lower levels of autofluorescence when compared to healthy tissue due to their increased metabolic activity. This increase in metabolic activity leads to the breakdown of the extracellular matrix as well as decreased concentration of Flavin adenine dinucleotide (FAD). FAD is a fluorophore and an endogenous compound that is excited at 430 nm. It absorbs blue light and autofluorescences green light. However, an area with a lower concentration of FAD would emit fewer green lights making the observation of FVL possible. Blue, green, and yellow wavelengths of light are absorbed best by hemoglobin. Due to increased number of blood vessels in tumors in comparison to normal healthy tissues, the tumorous tissue shows increased absorption and scattering of blue light. The thickening of the epithelium, increased vascularization, and dysplastic nuclei are all caused by the neoplastic development. These factors also lead to the absorption and/or the scattering of light.

Cancer is one of the leading causes of deaths globally; oral cancer is the 6^{th} most common cancers in Asia and Ranks top 3 of all cancers in India. Oral cancer occurs more often in people from the lower end of the socioeconomic scale and India has ⅓ of the world's oral cancer cases. Oral cavity cancers most of the times has a preceding PMDs. When identified at this stage by screening larger populations, their progression can be controlled which can reduce the incidence of oral cancer.

Regular screening and early diagnosis of cancers greatly increases the chances for successful treatment and enhance the healthcare outcomes. While fixed screening schedules are recommended in some types of cancer, some screening techniques are still evolving with ongoing research on methods for early cancer diagnosis including research to identify novel biomarkers as indicated in US patent 20110021370.

A number of point of care and portable devices are being developed and used in the clinics for such screening purposes. U.S. Pat. No. 9,535,068 discloses a point of care diagnostic test, device and disposables for determining a patient risk for oral cancer in the same visit that a sample is collected.

The VELscope is a device that increases the percent of cancers detected by revealing oral cancers that are indiscernible with the naked eye during standard examinations and cannot be used in a low resource setting due to economic constraints based on the cost of the device and the sensitivity of many such devices still remain low.

Various other patents U.S. Pat. No. 9,125,610, US20060241347, US20080318180 describe devices that can be used for oral cavity screening but lack multi-wavelength illumination (light emitting) sources which enable abnormal neovasculature and infections to be identified. These devices also lack image sensors and hence these devices cannot visualize the margins of the lesions nor any PMDs.

Some existing techniques and devices used for oral cancer detection with their advantages and disadvantages are as following:

| Process | | Description | Advantages | Disadvantages |
|---|---|---|---|---|
| Meta- | Toluidine | TB stains acidic | Sensitive | Shades of blue |
| chromasia | Blue (TB) | tissue components, | Economical | are open to |
| | | such as nucleic | Immediate | interpretation |
| | | acids. Can be used | results | Experiments are |
| | | most efficiently to | | not standardized, |
| | | confirm suspicious | | varying results |
| | | tumors as neo- | | Generally low |
| | | plastic, but does | | specificity |
| | | not have 100% | | Inflammation and |
| | | sensitivity, so can't | | trauma can cause |
| | | be relied onto show | | false-positives |
| | | lesions that are not | | |
| | | visible to the eye | | |
| | Lugol's | Iodine reacts with | Greater | Less sensitive than |
| | iodine | the glycogen in the | specificity | in identifying oral |
| | staining | cytoplasm, which | than TB | malignant |
| | | results in an observ- | Low cost | dysplastic lesions |
| | | able colour change. | | (than TB) |
| | | The enhanced | | |
| | | glycolysis in cancer | | |
| | | cells doesn't allow | | |
| | | the iodine start | | |
| | | reaction, so they | | |
| | | show up as pale, | | |
| | | while the normal | | |
| | | tissue is brown. | | |
| | Methylene | This dye, similar to | Similar to | Similar to |
| | Blue | TB, targets tissue | Toluidine | Toluidine Blue |
| | | with large amounts | Blue | |
| | | of nucleic acids, | | |
| | | which is found in | | |
| | | abnormal tissue. | | |
| | Rose Bengal | RB stains dead or | According to | Needs further |
| | (RB) | degenerated | one study, it | validation. |
| | | epithelial cells. Has | is capable of | Interpretation of |
| | | been used with | identifying | shading can limit |
| | | diagnosing ocular | lesions with | efficiency and |
| | | surface disorders. | oral squamous | accuracy |
| | | | cell carcinoma | Inflammation and |
| | | | (OSCC) | trauma can cause |
| | | | based on the | false-positives |
| | | | darkness of | |
| | | | the staining | |
| Tissue | VELscope | This device | hand-held | high initial costs |
| Absorbance | | observes the | easy to operate | moderate false |
| and Fluore- | | autofluorescence | simple to | positive rates |
| scence | | of blue light- | understand | as of now is an |
| | | absorbing fluoro- | takes ad- | adjunct aid, |
| | | phores to reveal | vantage of the | requires con- |
| | | abnormalities in the | disparity of | firmation by |
| | | tissue. Dark areas | endogenous | histopathological |
| | | indicate potential | fluorophore | analysis |
| | | malignancy, while | distribution in | the impact of |
| | | areas that show as | healthy and | hemoglobin on |
| | | green (the colour | cancerous | absorption and |
| | | emitted by fluoro- | tissue | fluorescence is yet |
| | | phore FAD) are | can detect | to be determined |
| | | shown as healthy. | cancers that | undetermined |
| | | | cannot be | impact of intensity |
| | | | seen during a | of light on |
| | | | routine | fluorescence |
| | | | examination. | loss of auto- |
| | | | | fluorescence also |
| | | | | can occur due to |
| | | | | inflammation and |
| | | | | melanin |
| | | | | pigmentation (not |
| | | | | only epithelial |
| | | | | abnormalities). |
| | ViziLite Plus | After being exposed | Improved the | Red lesions were |
| | | to 1% acetic acid, | visualization | harder to visualize |
| | | areas with abnormal | of white | Reflections |
| | | cells are visible | lesions | produced by |
| | | under bluish white | | chemiluminescent |
| | | LED light. | | light made |
| | | | | visualization |
| | | | | difficult |
| | | | | Insufficient |
| | | | | evidence for the |
| | | | | reliability of |
| | | | | ViziLite in early |
| | | | | detection of oral |
| | | | | malignancies |
| | Microlux/DL | Similar to Vizilite, | High | Poor discriminant |
| | | Microlux/DL | sensitivity | for inflammatory, |
| | | examines the oral | | traumatic, |
| | | cavity with blue- | | malignant diseases. |
| | | white LED light. | | Low specificity |
| | Orascoptic | Similar to Vizilite, | NA | NA |
| | DK | uses an acetic acid | | |
| | | rinse and blue-white | | |
| | | LED light. | | |
| | Photo- | Uses an acid called | NA | Rate of false |
| | dynamic | 5-ami-nolevulinic | | positive is high |
| | diagnosis | acid (ALA) that | | in patients with |
| | | induces proto- | | history of |
| | | porphyrin IX | | radiotherapy |
| | | (PPIX) to fluoresce | | Production of |
| | | in tissue. A 0.4% | | PPIX can also be |
| | | ALA rinse for 20 | | caused by reasons |
| | | minutes is able to | | other than |
| | | stimulate PPIX in | | cancerous tissue, |
| | | dysplastic and | | such as: |
| | | cancerous tissue, | | bacterial strains |
| | | which is then | | low levels of |
| | | excited to fluoresce | | glucose |
| | | by blue light. | | |
| Light | Raman | A technique that | Detected | Relatively weak |
| Spectro- | spectroscopy | distinguishes | cancers and | inelastic scattering |
| scopy and | | between normal | malignancy- | signals are difficult |
| Imaging | | and premalignant/ | associated | to capture |
| | | malignant tissue by | changes in the | Relatively slow |
| | | a vibrational | oral cavity | speed of |
| | | spectroscopy that | | spectrum |
| | | uses a laser in the | | |
| | | visible, near- | | |
| | | infrared, or near- | | |
| | | ultra-violet range | | |
| | | to cause inelastics | | |
| | | cattering. | | |
| | Narrow-band | This optical imag- | NA | NA |
| | imaging | ing technique is | | |
| | | based on the depth | | |
| | | of light penetration | | |
| | | and captures the | | |
| | | reflected light | | |
| Analysis | miRNA-184 | A greater amount of | Non-invasive, | Requires further |
| of Salivary | | microRNA-184 is | able to | validation in |
| bio marker | | present in oral | distinguish | large sub set of |
| | | abnormalities such | OSCC from | populations |
| | | as OSCC and oral | oral PMD | |
| | | PMD. | with dysplasia. | |
| | IL-6 | Inteerleukin-6 (IL- | Non-invasive | Periodontitis and |
| | | 6) level increases | | tobacco use can |
| | | with the severity of | | also affect levels |
| | | dysplasia. | | of IL-6 |
| OralCDx | OralCDx | Uses a specialized | Reports the | Not a final |
| Brush | Brush | brush that collects | presence or | diagnosis |
| Test | Test | samples of epithelial | absence of | |
| | | cells that are then | cellular | |
| | | scanned and | abnormalities | |
| | | analyzed micro- | | |
| | | scopically to | | |
| | | view abnormal | | |
| | | morphology. This | | |
| | | process is called | | |
| | | cytopathology. | | |

Hence, there is a need to develop a low cost device with increased sensitivity and specificity for screening oral cancer and the current invention, the “DR Oroscope”, can revolutionize early cancer detection.

### SUMMARY OF THE INVENTION

The present invention relates to a hand held intraoral medical device with a source of illumination emitting various specific wavelengths of light for screening of various oral diseases as abnormal tissue and dysplastic changes cannot be seen under a single source producing white light. An embodiment of the present invention is to provide a cost effective device to diagnose abnormalities in oral cavity, potentially malignant disorders, interdental caries, periodontal pockets and other such diseases. The device also assists in observation of vasculature in the lesions and possible infections in the oral cavity with the use of various wavelengths of light emitted from the light emitting source.

An embodiment of the present invention is described having a proximal end, the body and distal end or the tail of the device. The proximal end has alight source and a Charge Coupled Device Sensor based camera and optical filter mechanism, which is miniaturized for intraoral purpose. The distal end has the power supply mechanism. And the body which is hand-held has the heart of the system comprising of the microprocessor based circuitry with embedded software to capture and transmit the images/video; plurality of switches to control the wavelength, intensity of light and recording mechanism. The captured images/video are further processed using image/video processing software and displayed on a suitable device such as smart phone or smart tablet or computer for medical diagnosis to detect oral abnormalities.

### BRIEF DESCRIPTION OF DRAWINGS

The detailed description is described with reference to the accompanying figures. The left-most digit(s) of a reference number identifies the figure in which the reference number first appears. The same numbers are used throughout the drawing to reference like features and components.

FIG. 1: A top view of the device

FIG. 2: An oblique view of the device

FIG. 3: A top view of the PCB in the device

FIG. 4: A side view of the Printed Circuit board (PCB)

FIG. 5: A case of Oral submucous fibrosis observed under natural light (A), blue light extraorally (B) and compared with OralID (C)

FIG. 6: A case of pictures captured under natural light (A), filtered picture under natural light (B), picture captured under blue light (C), filtered picture captured under blue light (D)

FIG. 7: Block Level Description of the DR Oroscope system

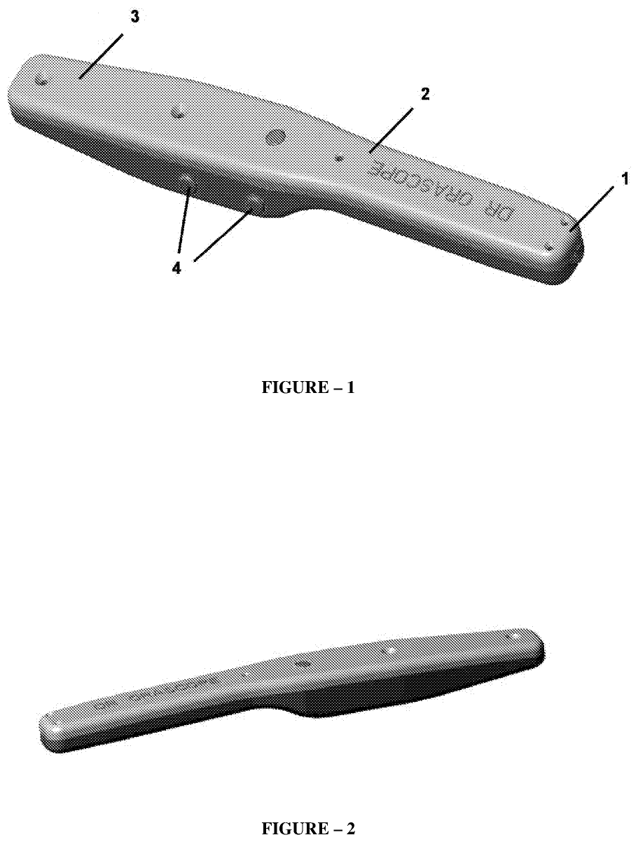

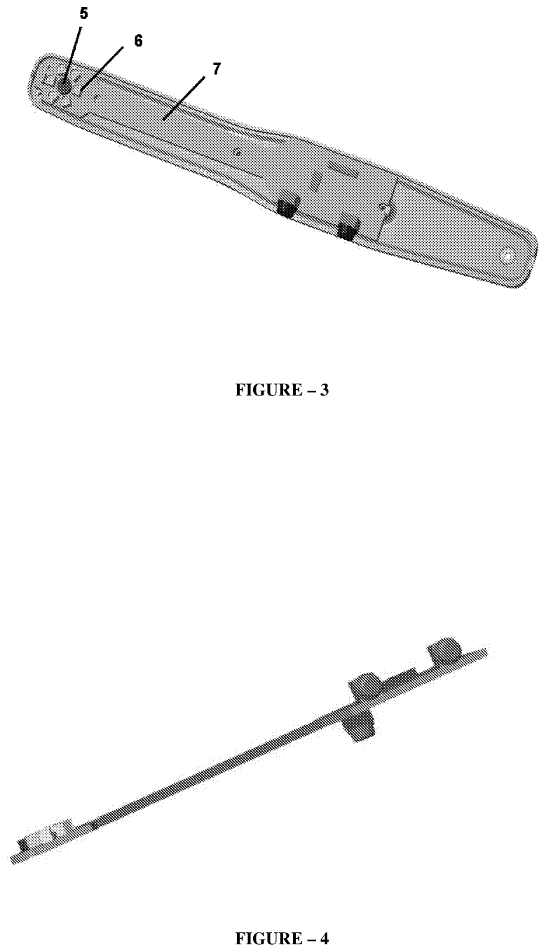

The drawings may show only those specific details that are pertinent to understanding the embodiments of the present invention. Further, skilled artisans will appreciate that elements in the drawings are illustrated for simplicity and may not have been necessarily been drawn to scale.

### DETAILED DESCRIPTION OF THE INVENTION

All terms used herein, including those specifically described below in this section, are used in accordance with their ordinary meanings unless the context or definition indicates otherwise. Also unless indicated otherwise, except within the claims, the use of “or” includes “and” and vice-versa. Non-limiting terms are not to be construed as limiting unless expressly stated (for example, “including” and “comprising” mean “including without limitation” unless expressly stated otherwise).

The present invention is a medical device designed portably and for intraoral use. The device is described having three sections:
a. the proximal end, or the head of the device (1);
b. the body of the device (2) and
c. the distal end or the tail of the device (3).

The proximal end of the device comprises a light source (6) and Charge Coupled Device sensor with Camera and Filter mechanism (5).

The light source (6) of the present embodiment has Light Emitting Diodes (LED) lights and supporting driving circuit can emit light of various wavelengths to illuminate the suspected area and the emitted light produces an optical signal which illuminates the target area. The Optional optical filters can be engaged to the light source to control the light parameters. The various wavelengths of light enables viewing of the oral cavity in day light or a room with light in contrary to the existing devices which can be used in dark rooms only for effective visualization. Various wavelengths of lights have observed to assist in specific diagnosis in the oral cavity. The present invention emits three different wavelengths of light; they are blue, red and green amber colour.

Blue light is used to detect potential malignant disorders and oral cancers based on the autofluorescence and loss of florescence properties of the tissue.

Red light is used to detect the infections that occur in various conditions.

Green amber light is used to detect vascularity as increased peripheral vasculature and central necrosis is a salient feature of malignancy. This light can also be used to detect the prognosis along with afore mentioned blue light.

Adjacent to or within the light emitting zone is a Charge Coupled Device (CCD) sensor (5) based camera, optical focus and optical filter mechanism (5) used for capture of images/video of the oral cavity. The CCD sensor (5) serves to capture the images by switching various light wavelengths, without moving the device thus helping in standardization. The intraoral device can be attached to communicate with any external devices such as smart mobile phones, smart tablets, computers or any other devices for storing and processing and displaying images/video. A filter can be fixed to the CCD sensor (5) which selectively blocks a particular wavelength of light from entering the sensor. This blocking of the optical inspection signal from the light source produces a different signal which is captured by the image sensors. The CCD sensor (5) captures the optical signal, and the intraoral device transfers the image data to application software on a smart device or computer which is capable of image processing like filtering and sharpening the images with low or excess light to enhance the clarity. This enhances the accuracy of capturing all the regions of the oral cavity including the margins of the lesion as well.

The device also comprises of optical filters which can be incorporated to the CCD sensor (5) camera mechanism. The filtering operation when done using image processing software externally enables the intraoral device to be more economical for manufacture and use in low and middle income countries.

The body of the device (2) comprises of plurality of switches (4) with plurality of positions to control the activation of the light source, wavelengths and intensities of the emitted light from the light source and recording mechanism (6). The body of the device connects the proximal (1) and the distal end (3) of the device and is the region served to hold the device by the user. The body of the device also consists of the Printed Circuit Board (7) which mechanically supports and electrically connects the electronic components of the device. The PCB (7) can be either rigid or flex-rigid or flexible made on any substrate. The body and the proximal end of the device can be designed to be made rigid or flexible.

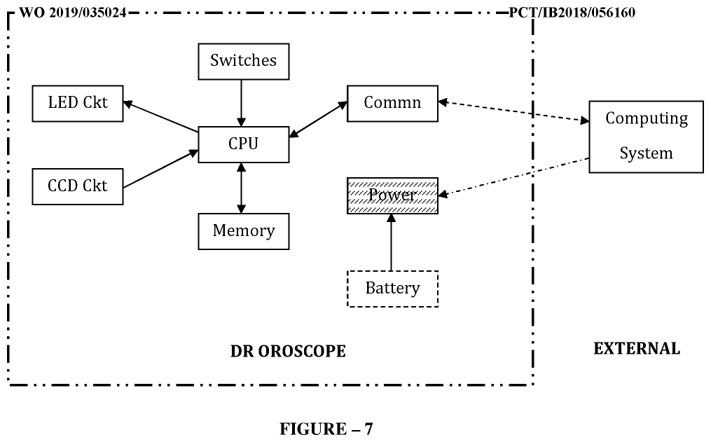

The body of the device contains the heart of the system as indicated in the block diagram FIG. 7. This can be understood by a person skilled in the art of designing electronics embedded systems and IoT (Internet of Things). The present embodiment is based on Microprocessor with Memory and Input/Output mechanisms. The embodiment can be alternately implemented on an ASIC or FPGA or Microprocessor or a combination thereof. The microprocessor executes embedded software stored in the Memory—PROM (Programmable Read Only Memory). There can be additional memory means (NAND Flash or SD Card) for storing the images within the intraoral device if required for use at a later time. The microprocessor interfaces the light source drivers, tunable optical filters, CCD Sensor circuit, Optical Focus mechanism, Input mechanism using Switches for light source profile and Data Transfer Communication means like USB port or Fiber Optic port or Bluetooth or WirelessLAN or cellular communication. The heart of the system need not be limited to the location in body (2) of the device. It can be located anywhere in the total intraoral device depending on the mechanical design provided the space and heat dissipation is taken care of. The device may have optional batteries for standalone power supply, else it can get power from external connected devices.

The distal end of the device (3) is used for the device's power-supply subsystem to power the PCB circuit and interface to an External Computing System like a smart device or a computer or any such related devices to capture and process the image/video. The distal end can have the power-supply subsystem with batteries OR a cable mechanism to provide the power when connected to a smart device or a computer or any such related device. The power supply subsystem need not be limited to the distal end (3) of the device and can be located anywhere in the total intraoral device mechanical design provided space and power supply dissipation is taken care of. The intraoral device can also be connected through a wired or wireless data communication network subsystem to the External Computing System like a smart device or a computer or any such related device. The present invention when connected through a data communications network (internet cloud) can transmit live and stored images to a smart device or a computer or any such related device which enables remote diagnosis by using “Dr Oroscope”.

The device of the present invention is made of materials like plastic or any polymer or thermoplastic kind of or resin or mixtures thereof. The term “Plastic” signifies material consisting of any of a wide range of synthetic or semi-synthetic organic compounds that are malleable and so can be molded into solid objects. It falls under a wide range of polymers like Polyamides or nylons, Polycarbonates, Polyesters, Polyethylene, Poly vinyl chloride, Poly vinylidene chloride, Acrylonitrile butadiene styrene or combinations thereof. The device can be a molded polymeric body (e.g., a thermoplastic material body) which can be made from a single layer of polymeric (plastic) material. The material used to form the container body can be selected so that the container body is visually transmissive and substantially impermeable at normal environmental pressures allowing for a suitable shelf life. Unlike conventional containers, the container body is not required to have the necessary characteristics to be autoclaved (e.g., withstand the high temperature, high pressure and steam of autoclaving). Examples of useful materials include, but are not limited to polycarbonate, polyolefin such as polypropylene (PP), polyethylene (PE), or cyclic olefin (COC), polyester such as polyethylene terephthalate (PET) or polyethylene napthalate (PEN), polyamide (nylon), or other well-known materials in the plastics art. Within Polyethylene, LDPE (Low density poly ethylene) as well as HDPE (High density polyethylene) both can be used as the polymeric body. Amorphous plastics such as amorphous nylon exhibit high transparency may also be suitable.

This device works based on fluorescence activity and uses light with various wavelengths for viewing the oral cavity by penetrating the epithelial tissues and reaching the stroma through the basal layer. This will allow the user to observe the suspicious lesions in different colours as the green light enables visibility of vasculature in the lesions which is increased in cancerous tissue and the red lights enables visibility of any infections in the oral cavity. The device of the present invention has advantages over the other devices on following points:
1. Portability: Intraoral portable device
2. Connectivity: Can be connected to any smart deice like mobile phone or tablet; computer and related device. The device can take power from these devices for operation which further enables its use in low resource settings. The images can be transferred to these external devices either through a wireline connection like USB or optical fiber cable OR wireless connection through WirelessLAN or Bluetooth or related programs or through data communications network to provide telemedicine based services.
3. Visibility: The data from the intraoral device camera when transferred to the smart devices like mobile phones, computer and related devices, enables larger visibility of the oral cavity, which further facilitates better examination and screening. The camera integrated into the device assists in capturing the images by the user while performing the examination.
4. Reproducibility: High and repeated photographs can be taken
5. Affordability: Cost of manufacturing the device is low and suitable for use in low and middle income countries
6. Acceptability: Is higher as it is a non-invasive technique of examination
7. Accuracy: Increased accuracy as different wavelengths and intensities of light are produced.
8. Accessibility: Accessibility to every corner of the mouth as intraoral device and can be used by any medical and paramedical staff in any place

Experimentation:

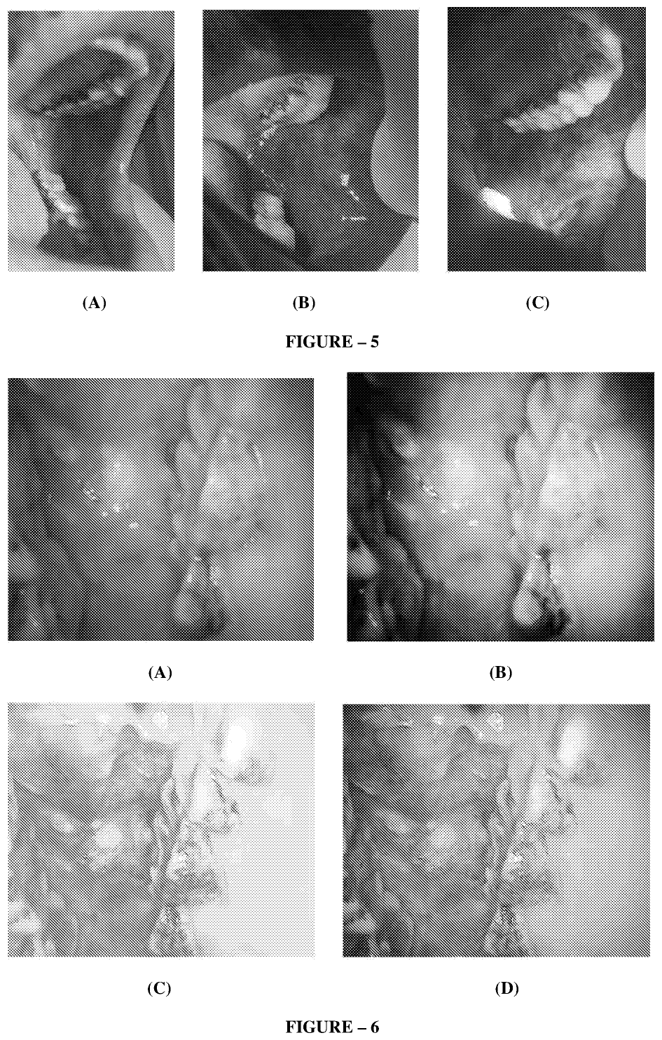

A pilot screening was performed by screening 100 patients for oral cavity diseases. The personal information, social information, prior medical history, habits of the patients and additional information for the visit to clinician were recorded. Medical history collected assists in if the intraoral lesion might be a part of a systemic disease and the course of treatment required to be provided to the patient. A thorough examination was conducted on the patients with the present invention, “DR Oroscope” system. Potentially malignant lesions observed in each patient were examined twice: The first examination consisted screening of the oral cavity by the device, DR Oroscope followed by a biopsy of the lesion for confirmation of pathology. When examined by using DR Oroscope, the examination is conducted by using blue light, green light and red light, sequentially one after the other to observe the entire oral cavity of the patient under all these wavelengths of light. FIG. 5 indicates a case examined under natural light (A), using blue light of DR Oroscope (B) and another commercially available device, OralID (C). The neovasculature and lesion is visibly clearer when observed with the use DR Oroscope. FIG. 6 indicates a case examined under natural light (A) and blue light (C) along with the images viewed by applying the filters under white light (B) and blue light (D). The filters applied herein enhance the image for precise diagnosis.

Statistical Analysis:

The malignancy of each detected lesion was determined and 26 cases comprising of 11 Lichen planus, 7 leukoplakias. 5 squamous cell carcinomas, 3 Oral sub mucous fibrosis were identified as high-risk lesions.

Sensitivity, specificity, accuracy, positive predictive value, and negative predictive value were calculated using the following formulas:

Sensitivity=[true positives/(true positive+false negative)]×100

Specificity=[true negatives/(true negative+false positives)]×100

Accuracy=Sensitivity+Specificity

Predictive value for a positive result (PV+)=[true positive/(true positive+false positive)]×100

Predictive value for a negative result (PV−)=[true negatives/(true negatives+falsenegatives)]×100.

Patient Distribution:

The sample distribution was statistically analyzed. The sample consisted of 50 males and 50 females with a mean age of 37-40 years.

The results show the occurrence and gender wise distribution of potential malignant lesions. Clinically undifferentiated lesions were higher in females (90-94%) compared to males (86-90%), while clinically differentiated lesions were higher in males than females (9-10%).

There were no statistically significant differences between male and female patients with potentially malignant lesions.

Subjects' distribution according to the types of lesions is observed as indicated in FIGS. 5 and 6. Data indicated that Traumatic ulcer had the highest incidence (26.8%) and or pemphigusvulgaris (1.4%) was the least common.

In connection with the foregoing embodiments, additional variations will now be apparent to persons skilled in the art. Various modifications and variations to the above described device can be made without departing from the scope of the invention.

From the foregoing it will be understood that the embodiments of the present invention described above are well suited to provide the advantages set forth, and since many possible embodiments may be made of the various features of this invention and as the device herein described may be varied in various parts, all without departing from the scope of the invention, it is to be understood that all matter hereinbefore set forth or shown in the accompanying drawings is to be interpreted as illustrative and that in certain instances some of the features may be used without a corresponding use of other features, all without departing from the scope of the invention.

Further the present invention has to be illustrated in the different figures. The following specific and non-limiting steps for functioning need to be construed as merely illustrative, and do not limit the present disclosure in any way whatsoever.

1. A hand held medical device used for intraoral screening of the oral cavity comprising of:
a. a power supply source;
b. a light source for illumination, wherein the light source includes a plurality of light emitters configured to emit light for selected wavelengths and selected intensities;
c. an input mechanism for selecting the profile of light sources, and selecting wavelength and intensity of light sources;
d. a computing subsystem to interface the input and output subsystems and memory subsystem;
e. an image sensing and capturing mechanism for image or video;
f. data communication subsystem to transfer the data from this device to an external computing system;
g. an electronic wiring mechanism to connect the computing subsystem to a light source means, input means, image sensing and capturing means, data communication means and power supply;
h. Mechanical chassis to encompass the above.

2. A hand held medical device of claim 1, wherein the power supply source is selected from one of an external supply source or built-in battery or an energy scavenging device.

3. A hand held medical device of claim 1, wherein the light source comprises a plurality of light emitters.

4. The light source of claim 1 wherein the light source comprises of a plurality of light emitters, an optional optical filter for the light emitters, able to provide a specific wavelength and specific intensity.

5. The light source of claim 1 is preferably implemented using LED.

6. The input mechanism of the hand held medical device of claim 1 comprising of plurality of switches with plurality of positions per switch to select a desired profile of light sources for selecting wavelengths and intensity of light.

7. The input mechanism of the hand held medical device of claim 1 comprising of a wireless method to select a desired profile of light sources for selecting wavelengths and intensity of light.

8. The input mechanism of the hand held medical device of claim 1 comprising of a wireline method to select a desired profile of light sources for selecting wavelengths and intensity of light, provided that the device of claim 1 is connected to an external system using a cable.

9. The computing system of the hand held medical device of claim 1 comprising of a Central Processing Unit (CPU), memory subsystem, Input/Output Subsystem and interfaces the other subsystems in claim 1.

10. The image sensing and capture mechanism of the hand held medical device of claim 1, comprises an image detector with optical focus mechanism, and an optional optical filter mechanism on the image sensor.

11. The image sensor of claim 1 is implemented using CCD.

12. The optical filter mechanism of claim 4 can be moved selectively into and out of said optical pathway in response to the input means.

13. The data communication subsystem of the hand held medical device of claim 1, can communicate via wire using electrical interface, having preferably high speed data rates, using any of the standard protocols from the group of USB, Ethernet, Firewire.

14. The data communication subsystem of the hand held medical device of claim 1, can communicate via wire using fiber optic interface.

15. The data communication subsystem of the hand held medical device of claim 1, can communicate wirelessly using any of the standard protocols from the group of Bluetooth, IrDA, WirelessLAN, WirelessMAN, WirelessPAN, Cellular Communication.

16. The image sensing and capture mechanism of the hand held medical device of claim 1, comprises an image detector with optical focus mechanism, and an optional optical filter mechanism on the image sensor, can store the data in the device using the memory subsystem, or transfer the data out via wire using electrical interface, having preferably high speed data rates, using any of the standard protocols from the group of USB, Ethernet, Firewire.

17. The electronic wiring mechanism of the hand held medical device of claim 1, can be a rigid or flex-rigid or flexible PCB.

18. The mechanical chassis of the hand held medical device of claim 1, can be a rigid or flexible at the proximal end and the body of the device.

19. A hand held medical device of claim 1 further comprises at least one selected illumination wavelength located in an illumination light path from the device of claim 1 to the suspected region and configured to selectively allow at least one desired wavelength of the light being transmitted out of the target region.

20. A portable hand held medical device of claim 1 comprising of light source and camera mechanism for carrying out several separate or concurrent purposes by a user for intraoral examination and/or treatment procedure carried out within the oral cavity.

21. A method of conducting remote diagnosis for providing telemedicine services by a hand held medical device of claim 1 comprising:
a. enabling the user of the device of claim 1 to perform examination,
b. connecting the user of the device of claim 1 to a experienced medical practitioner through data communications network for transferring the data from the user to the medical practitioner,
c. transfer of the live images or video of the patient by the user of the device of claim 1 to the medical practitioner through data communications subsystem,
d. transfer of the stored images or video of the patient by the user of the device of claim 1 to the medical practitioner through data communications subsystem.

22. A method of screening oral cavity and diagnosing oral cancer and potentially malignant disorders by using a hand held portable intraoral device wherein the hand held portable device comprises of a light emitting source which can emit light of various wavelengths and intensities; an image sensor based camera with optional filter mechanism; and a data communication subsystem to transfer the image or video data to an image or video processing, data recording and displaying application software on an External Computing System.

23. The light source of claim 3 wherein the light source comprises of a plurality of light emitters, an optional optical filter for the light emitters, able to provide a specific wavelength and specific intensity.

24. The optical filter mechanism of claim 10 can be moved selectively into and out of said optical pathway in response to the input means.

25. The diagnosis of claim 22 based on
a. one image or video generated using one profile of light source, further processed using image or video processing software,
b. at least two images or videos generated using different profiles of light sources, and further processed using image or video processing software.
